(19) United States
(12) Patent Application Publication
(10) Pub. No.: US 2003/0144674 A1
(43) Pub. Date: Jul. 31, 2003

(54) Manipulator for fixing a surgical needle

(76) Inventors: Thierry Loubens, Saint-Didier au Mont d'Or (FR); Lionel Riou, Lyon (FR); Antoine Watrelot, Lyon (FR)

Correspondence Address: GREENBLUM & BERNSTEIN, P.L.C., 1950 ROLAND CLARKE PLACE, RESTON, VA, 20191, US

(21) Appl. No.: 10/257,389

(22) PCT Filed: Mar. 29, 2001
(86) PCT No.: PCT/FR01/00948

(51) Int. Cl. A61B 17/04

(52) U.S. Cl. 606/148

(57) ABSTRACT

The invention concerns a manipulator for fixing a surgical needle integral with a suture, comprising a sleeve (4) wherein is stressed in a substantially elongated position a surgical needle (2), a stem (13) which penetrates into the sleeve (4) for extracting said needle (2) and angular indexing means (19) which consist in a configuration of the inner bore (5) of the sleeve (4) according to a profile which enables during extraction of the surgical needle (2) from the sleeve (4) to direct it into a specific and constant position relative to the main axes of said sleeve.

The present invention relates to an applicator for positioning a surgical needle which is integral with its suture thread in the operating site, and more particularly to improvements permitting angular indexing of the position of the surgical needle in relation to said applicator.

The applicator according to the present invention is intended for endoscopy procedures in order to cooperate, for example, with a trocar of small diameter so as to bring the surgical needle to the operating site and release it there.

The patent application EP 0 554 361 has disclosed an applicator comprising a cylindrical cannula integral, at one of its ends, with grips so that it can be held by the surgeon, with a hollow sleeve which at one of its ends retains the surgical needle, while the opposite end is integral with a grip allowing the surgeon to slide the sleeve inside the cannula in order to release the needle in the operating site. The free end of the hollow sleeve retaining the surgical needle is in the form of a mandrel comprising clamping jaws which deform axially on account of the difference in diameter between the internal diameter of the cannula and the external diameter of the mandrel, so that said jaws will clamp tight about the needle and retain it inside the cannula.

Extracting the mandrel from the cannula by sliding the sleeve inside the latter makes it possible to release the clamping pressure on the jaws, so that the surgeon, with the aid of a pincer, can recover the surgical needle maintained in position in said mandrel.

It will be noted that the device for retaining the surgical needle in the applicator allows the latter to be held in a defined angular position relative to the hollow sleeve, but not relative to the cannula, because said sleeve is able to turn inside said cannula.

The rotation of the sleeve inside the cannula means that the needle emerges in a position which is random and always different relative to the cannula, obliging the surgeon to reposition the needle in the operating site so as to be able to make the first suture point.

The patent FR 97 07681 belonging to the Applicant also discloses an applicator for positioning a surgical needle in the operating site.

The applicator described in the patent FR 97 07681 comprises a sleeve in which a needle is stressed in a substantially elongate position, a rod which penetrates into the sleeve for extracting said needle, and retention means with which it is possible, before extraction of the needle, to maintain the rod inside the sleeve in a defined position so that the free end of said rod bears against the needle.

In addition, the applicator comprises, in the inner part of the sleeve, tensioning means with which it is possible to keep the suture thread taut during extraction of the needle.

The applicator according to the present invention is for remedying the disadvantages of the applicator described in, the patent application EP 0 554 361 by allowing the surgeon to know in which position the surgical needle will emerge in the operating site.

The object of the applicator according to the present invention is to improve the applicator described in the patent FR 97 07681 in order, on the one hand, to allow the surgical needle to emerge in a defined and constant position relative to the sleeve and, on the other hand, to limit the travel of the rod for extraction of the needle relative to the sleeve.

The applicator for positioning a surgical needle which is integral with a suture thread according to the present invention comprises a sleeve in which the surgical needle is stressed in a substantially elongate position, a rod which penetrates into the sleeve for extracting said needle, and angular indexing means which consist of a shaping of the sleeve at one of its free ends according to a profile which, during extraction of the surgical needle from the sleeve, makes it possible to direct said needle into a defined and constant position relative to the main axes of said sleeve.

The applicator for positioning a surgical needle which is integral with a suture thread according to the present invention comprises angular indexing means consisting of a shaping of the external diameter D1 of the sleeve such that the inner bore of said sleeve has an oblong inner profile whose width (e) is less than the external diameter D1, while its height H is similar to that of the external diameter D1 so as to make it possible, during extraction of the surgical needle from the sleeve, to direct said needle into a defined and constant position relative to the main axes of said sleeve.

The applicator for positioning a surgical needle which is integral with a suture thread according to the present invention comprises an external diameter D1 of the sleeve which is deformed laterally so that the end has a profile made up of two parallel flat parts connected to the cylindrical profile of the sleeve via inclined portions.

The applicator for positioning a surgical needle which is integral with a suture thread according to the present invention comprises an inner bore which has, between its oblong inner profile and its internal circular diameter, an inclined portion originating from the outer inclined portions.

The applicator for positioning a surgical needle which is integral with a suture thread according to the present invention comprises an inner oblong profile which ends in an inner bevel making it possible to position and more easily introduce the needle inside the inner bore of the sleeve when it is necessary to retract said needle from the operating site.

The applicator for positioning a surgical needle which is integral with a suture thread according to the present invention comprises an oblong inner profile communicating with the inner bore of the sleeve and which has, at the level of the inner bevel, a circular profile of external diameter D2 which is smaller than that D1 of said sleeve, giving the free end what is substantially a trumpet shape.

The applicator for positioning a surgical needle which is integral with a suture thread according to the present invention comprises a sleeve in which the profile of the internal diameter is obtained by shaping a metal tube.

The applicator for positioning a surgical needle which is integral with a suture thread according to the present invention comprises a sleeve in which the profile of the internal diameter is obtained upon molding of said sleeve.

The applicator for positioning a surgical needle which is integral with a suture thread according to the present invention comprises a rod which has a head integral with flexible tongues making it possible to limit the travel of the rod relative to the sleeve.

The applicator for positioning a surgical needle which is integral with a suture thread according to the present invention comprises retention means which consist of a plate of oblong shape, of which one of the faces is integral with two clamps which cooperate respectively with the rod and the sleeve.

The applicator for positioning a surgical needle which is integral with a suture thread according to the present invention comprises a sleeve which has a cylindrical head provided on its outer face with a circular impression which cooperates with the clamp of the plate, while the rod comprises a head equipped with an impression about which the clamp of said plate clicks into place in such a way as to maintain a constant distance L between the heads so as to ensure that the free end of the rod is on the one hand in contact with the needle and on the other hand at a distance L′ from the free end of the sleeve.

The applicator for positioning a surgical needle which is integral with a suture thread according to the present invention comprises a sleeve which has, starting from the end, and in the direction of the head, two grooves which are diametrically opposite one another and parallel to the longitudinal axis of the applicator.

The applicator for positioning a surgical needle which is integral with a suture thread according to the present invention comprises grooves which have a V-shaped profile, of which the most open base, or widest base, is directed toward the end.

The applicator for positioning a surgical needle which is integral with a suture thread according to the present invention comprises grooves which have a depth decreasing along the periphery of the sleeve, so that the deepest level is toward the free end and the most open base of the V.

The applicator for positioning a surgical needle which is integral with a suture thread according to the present invention comprises an end of the sleeve having a slit arranged perpendicular to the grooves in such a way that each groove cooperates with said slit.

The applicator for positioning a surgical needle which is integral with a suture thread according to the present invention comprises a slit which forms a hollow inside the sleeve which communicates with the inner bore.

The following description in which reference is made to the attached drawings, given as nonlimiting examples, will provide a better understanding of the invention, of its characteristics, and of the advantages it will afford:

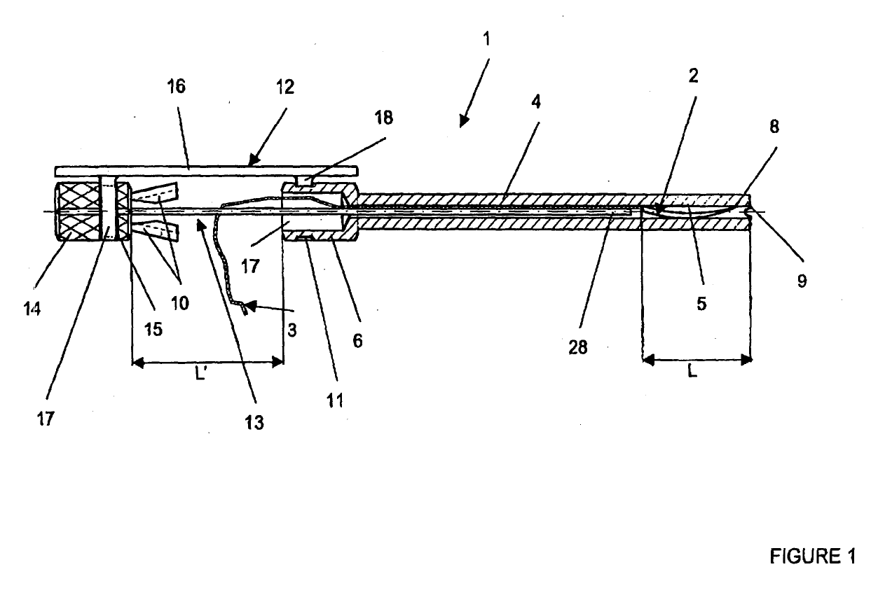

FIG. 1 is a cross section illustrating the applicator for positioning a needle according to the present invention.

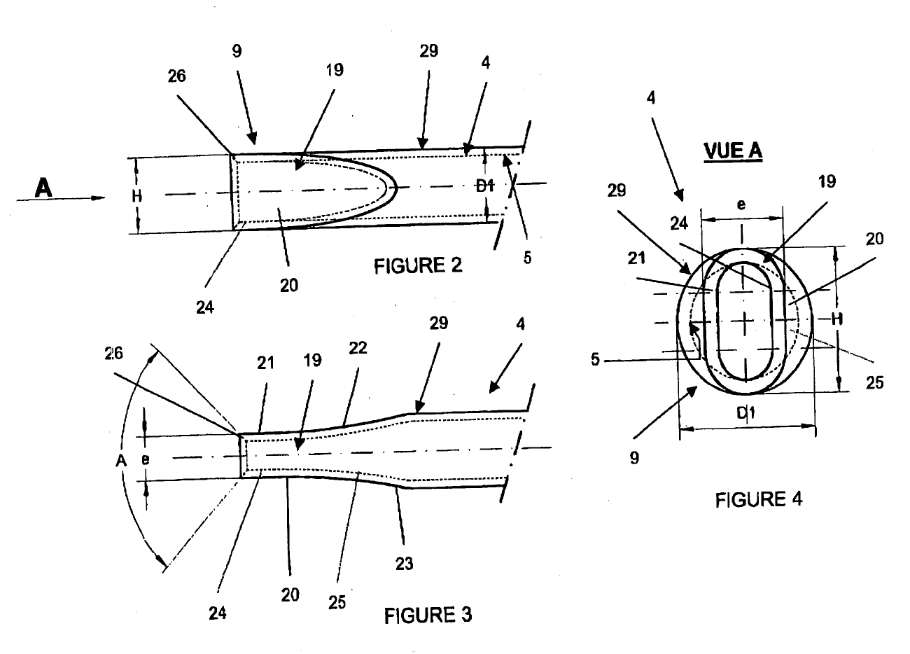

FIGS. 2 through 4 are views representing the angular indexing means for positioning the surgical needle during extraction from the sleeve of the applicator according to the present invention.

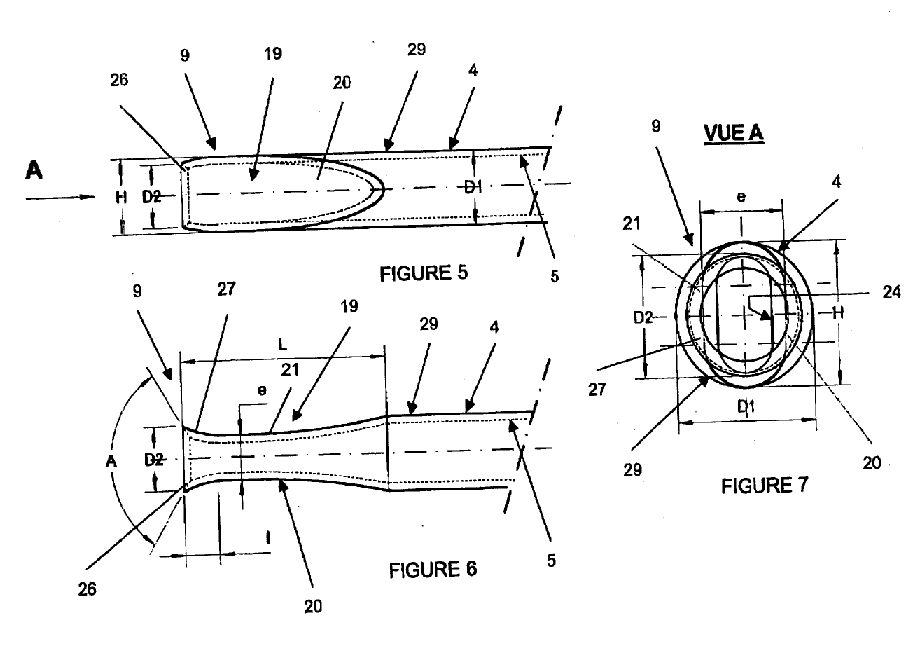

FIGS. 5 through 7 are views showing a variant of the angular indexing means in FIGS. 2 through 4.

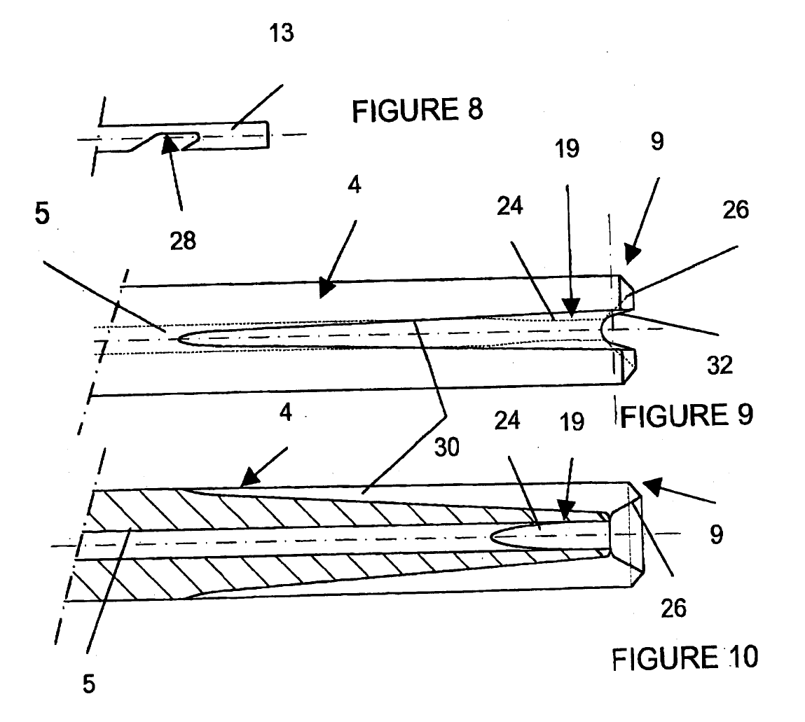

FIG. 8 is a view illustrating in detail the hook-shaped. profile of the rod constituting the needle-pusher of the applicator according to the present invention.

FIGS. 9 and 10 are views showing the free end of the sleeve, obtained directly by molding.

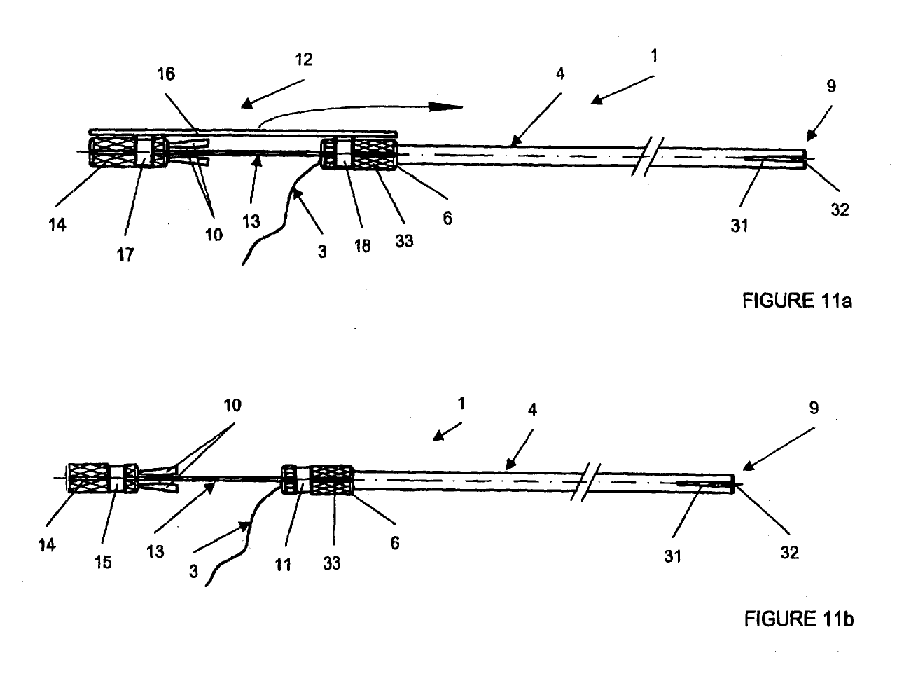

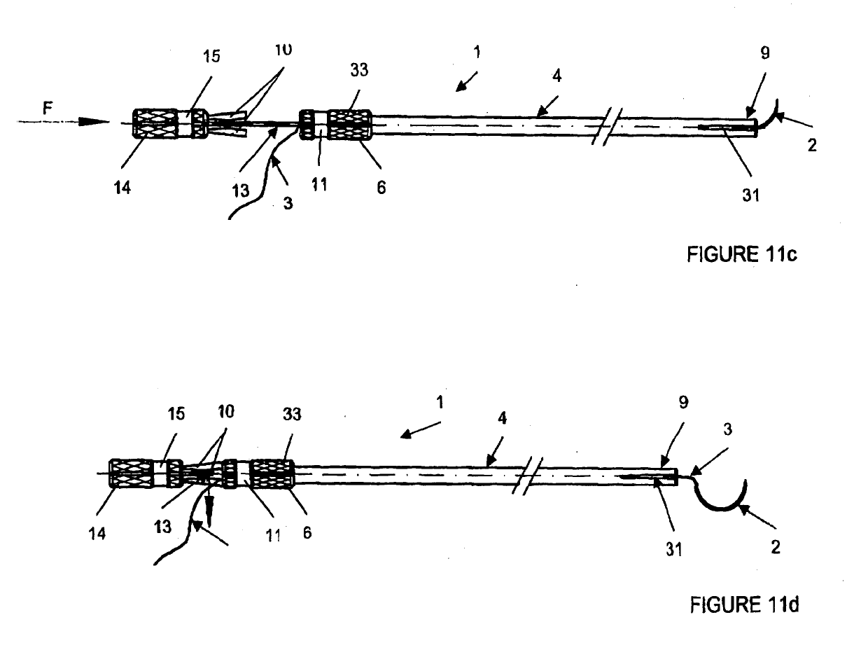

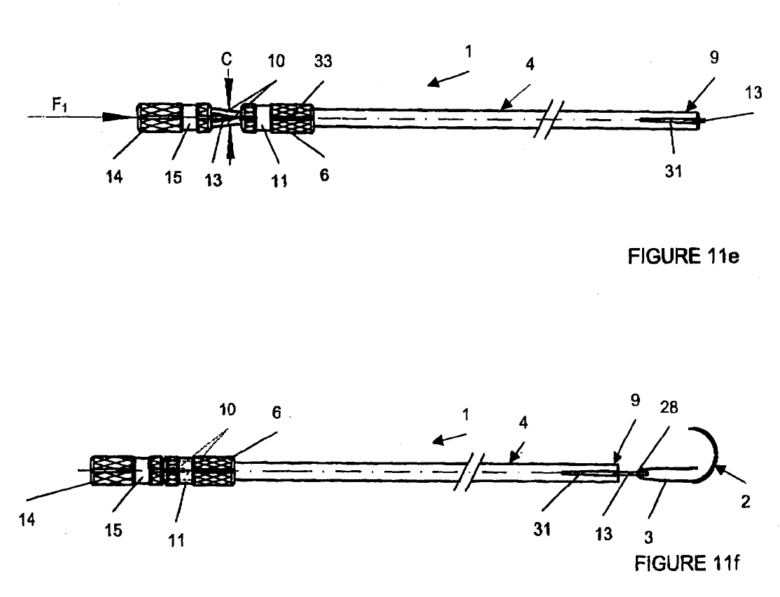

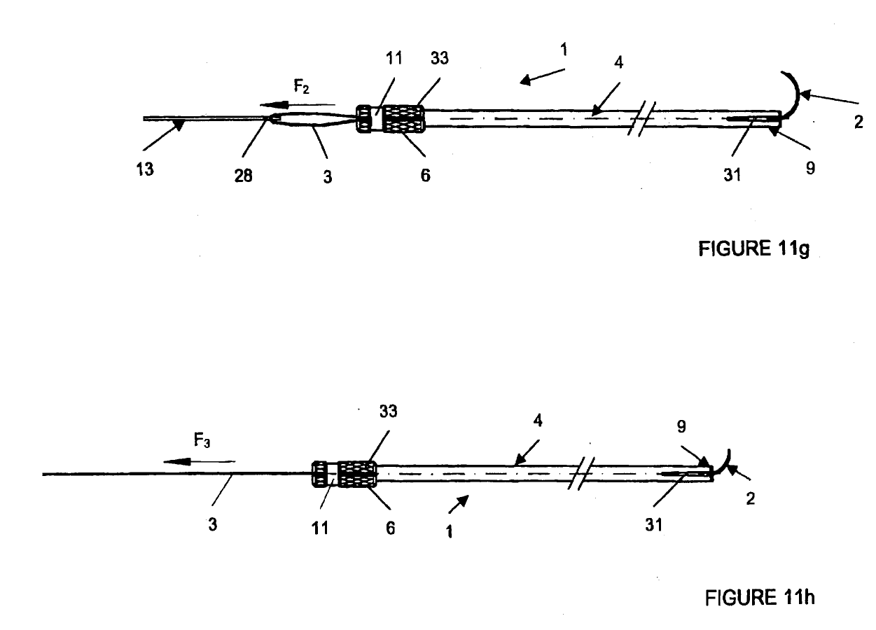

FIGS. 11a through 11h are views showing the various steps in the positioning and use of the applicator according to the present invention.

In FIG. 1, an applicator 1 has been shown which is similar to that described in the patent FR 97 07681 and which permits positioning of a surgical needle 2 integral with a suture thread 3 in the surgical site by way of a trocar (not shown) and which has been placed beforehand through the wall of a patient.

The applicator 1 comprises a cylindrical sleeve 4 having along its entire length, and parallel to its longitudinal axis, an inner bore 5 in which the needle 2 and its suture thread 3 are accommodated.

The sleeve 4 has, at one of its ends, a cylindrical head 6 having a diameter greater than that of the rest of the body of said sleeve. Formed inside the head 6, coaxially with respect to the bore 5, there is a cylindrical seat 7 which has a diameter greater than that of said bore.

The seat 7 communicates with the inner bore 5 in such a way that the suture thread 3 integral with the needle 2 can emerge outside the sleeve 4.

The needle 2 is in fact arranged in the inner bore 5 of the sleeve 4 in such a way that its tip 8 is directed toward the free end 9 and away from that bearing the head 6.

The outer wall of the head 6 has a circular impression 11 cooperating with retention means 12 which are intended to hold a rod 13 or needle-pusher in a defined position relative to the sleeve 4.

At one of its ends, the rod 13 is integral with a head 14 comprising, on its outer face, a circular impression 15 intended to cooperate with the retention means 12.

At its free end remote from the head 14, the rod 13 comprises a hook 28 intended to recover the suture thread 3 from the needle 2 (FIG. 8).

The head 14 comprises, in its continuation and on either side of the rod 13, flexible tongues 10 in the form of clamps which in the normal state constitute an abutment for limiting the displacement travel of the rod 13 relative to the sleeve 4.

The rod 13 is introduced into the sleeve 4 so as to come to bear against the needle 2. The rod 13 passes through the seat 7 and the inner bore 5 in such a way that the suture thread 3 is disposed between said rod and the wall of said bore.

The retention means 12 make it possible to maintain the rod 13 relative to the sleeve 4, in such a way that the free end of the rod 13 remote from the head 14 is always bearing against the needle 2.

The retention means 12 consist of a plate 16 joined integrally, on one of its faces, to two clamps 17 and 18 forming fixation clips. Each clamp 17 and 18 cooperates respectively with the impression 15 of the head 14 integral with the rod 13, and the impression 11 of the head 6 of the sleeve 4.

The position of the two clamps 17 and 18 on the plate 16 makes it possible to maintain a constant distance L between the head 14 of the rod 13 and the head 6 of the sleeve 4.

This distance L ensures that the free end of the rod 13, that is to say the end remote from the head 14, is on the one hand in contact with the needle 2 and on the other hand at a distance L′, identical to the distance L, from the free end 9 of the sleeve 4.

FIGS. 2 through 4 show the profile of the free end 9 of the sleeve 4 when the latter is formed by a metal tube 29 about which a cylindrical plastic envelope (not shown) is overmolded.

The free end 9 comprises means 19 for angular indexing of the surgical needle 2 relative to the main axes of said sleeve.

The angular indexing means 19 consist of a shaping of the external diameter D1 of the sleeve 4 such that the inner bore 5 of said sleeve has a particular profile making it possible, during extraction of the surgical needle 2, to direct the latter into a defined and constant position relative to the main axes of the sleeve 4.

The external diameter D1 of the sleeve 4 is deformed laterally so that the end 9 has a profile made up of two parallel flat parts 20 and 21 which are connected to the cylindrical profile of the sleeve via inclined portions 22 and 23.

The deformation of the diameter D1 the sleeve 4 leads to the deformation of the inner bore 5 which has an oblong inner profile 24 whose width e is smaller than the external diameter D1, while its height H is similar to that of the external diameter D1.

The inner bore 5 has, between its oblong inner profile 24 and its internal circular diameter, an inclined portion 25 originating from the outer inclined portions 22 and 23.

The inner inclined portion 25 makes it possible to direct the needle 2 in the sense of the oblong inner profile 24, so that it always emerges from the sleeve 4 in a defined and constant position.

The inner oblong profile 24 ends in an inner bevel 26 inclined by an angle A which is directed toward the outside of the sleeve 4 and making it possible to position and more easily introduce the needle 2 inside the inner bore 5 of the sleeve 4 when it is necessary to retract said needle from the operating site.

FIGS. 5 through 7 show a variant of the profile of the free end 9 of the sleeve 4 when the latter consists of a metal tube 29 around which a cylindrical plastic envelope (not shown) is overmolded.

The angular indexing means 19 have a profile similar to that described before in FIGS. 2 through 4, but permit better introduction of the needle 2 inside the inner bore 5 for its extraction from the operating site.

It will be noted that the oblong inner profile 24 communicating with the inner bore 5 of the sleeve 4 has, at the level of the inner bevel 26, a circular profile 27 of external diameter D2 which is smaller than that D1 of said sleeve, giving the free end 9 what is substantially a trumpet shape.

The circular profile 27 facilitates the introduction of the needle 2 inside the inner bore 5 of the sleeve 4 during its extraction from the operating site, because the surface of introduction is greater than that of the previously described oblong profile 24.

FIGS. 9 and 10 show the sleeve 4 which is made integrally of plastic without the use of the metal tube 29.

In this case, the sleeve 4 comprises an inner bore 5 whose free end 9 has indexing means 19 obtained upon molding of said sleeve.

The angular indexing means 19 consist of a shaping of the internal diameter of the bore 5 of the sleeve 4 during its molding, so as to present a particular profile which, during extraction of the surgical needle 2, makes it possible to direct said needle into a defined and constant position relative to the main axes of the sleeve 4.

Thus, the inner bore 5 has an oblong inner profile 24 similar to that previously described for the metal tube 29.

The shaping of the inner bore 5 makes it possible to direct the needle 2 in the sense of the oblong inner profile 24 so that it always emerges from the sleeve 4 in a defined and constant position.

The inner oblong profile 24 ends in an inner bevel 26 directed toward the outside of the sleeve 4 and making it possible to position and more easily introduce the needle 2 inside the inner bore 5 of the sleeve 4 when it is necessary to retract said needle from the operating site.

The sleeve 4 comprises, starting from the end 0.9, and in the direction of the head 6, two grooves 30 and 31 which are diametrically opposite one another and parallel to the longitudinal axis of the applicator 1.

Each groove 30 and 31 has a V-shaped profile of which the most open base, or widest base, is directed toward the end 9. Moreover, each groove 30 and 31 has a depth which decreases along the periphery of the sleeve, so that the deepest level is toward the free end 9 and the most open base of the V.

The end 9 of the sleeve 4 comprises a slit 32 arranged perpendicular to the grooves 30, 31 in such a way that each groove cooperates with said slit. The diameter of the slit 32 is substantially identical to the dimensions of the most open base of the V forming the profile of each groove 30, 31.

The slit 32 forms a hollow inside the sleeve 4 which communicates with the inner bore 5 in which the rod 13 and the needle 2 integral with the suture thread 3 slide.

Finally, the head 6 of the sleeve 4 comprises, respectively in the alignment of the grooves 30, 31, an impression 33 forming a marker for the surgeon to position the slit 32 and the grooves in the operating site (FIGS. 11a through 11h).

The sleeve 4 equipped with the grooves 30, 31, the slit 32 and the impressions 33 allows the surgeon to knot the suture thread 3 outside the human body.

FIGS. 11a through 11h show the different steps for using the applicator 1 according to the present invention.

The applicator 1 is ready to be used. The surgeon introduces it into the operating site (FIG. 11a).

Once the applicator 1 has been introduced by the surgeon into the operating site by way of a trocar (not shown), the surgeon then withdraws the retention means 12 in order to release the rod 13 of the sleeve 4 (FIG. 11b).

The surgeon presses on the head 14 of the rod 13 in the direction of the arrow F in order to displace said rod inside the bore 5, in order to have the needle 2 emerge from the sleeve 4 in a defined position depending on the position of the indexing means 19 (FIG. 11c). In this position, the surgeon can make his first suture point by using the sleeve 4 to cause the needle 2 to penetrate into the tissues to be sewn.

Alternatively, the surgeon can continue to push the rod 13 so that the tongues 10 come to bear against the head 6 of the sleeve 4. When the tongues 10 are in contact with the head 6, the surgeon is certain that the needle has completely emerged from the sleeve 4 (FIG. 11d) In this position, the surgeon recovers the needle 2 with the aid of a needle-holder in order to make his first point and the subsequent ones. Moreover, the tongues 10 bearing against the head 6 of the sleeve 4 prevent the free end of the rod 13 from penetrating into the operating site and damaging the neighboring tissues.

To recover the needle 2 from the operating site, the surgeon compresses the flexible tongues 10 in the direction c and pushes the head 14 of the rod 13 in the direction of the arrow F1. The deformation of the tongues 10 allows them to penetrate into the seat 7 of the head 6 and to prolong the displacement of the rod 13 inside the inner bore 5 of the sleeve 4 (FIG. 11e)

Thus, the surgeon can push the head 14 of the rod 13 so that the latter comes to bear against the head 6 of the sleeve 4. The contact between the two heads 6 and 14 of the applicator 1 assures the surgeon that the free end of the rod 13 provided with the hook 28 is inside the operating site. The surgeon recovers the suture thread 3 of the needle 2 with the aid of the hook 28 (FIG. 11f).

The surgeon then pulls the rod 13 in the direction of the arrow F2 in order to extract it from the sleeve 4 and recover the suture thread 3 at the level of the head 6. In this position, the needle 2 is introduced slightly into the sleeve 4 at the level of its end 9 (FIG. 11g).

The surgeon finally pulls on the suture thread 3 in the direction of the arrow F3 in order to introduce the needle 2 completely inside the sleeve 4 (FIG. 11h).

The applicator 1 according to the present invention can be used to form suture knots inside or outside the body, depending on the surgeon's suturing technique.

It must also be understood that the above description has been given solely by way of example and that it does not in any way limit the scope of the invention, and it will be possible to replace the described embodiment details with any other equivalent ones without departing from said scope of the invention.

1. An applicator for positioning a surgical needle (2) which is integral with a suture thread (3), characterized in that it comprises a sleeve (4) in which the surgical needle, (2) is stressed in a substantially elongate position, a rod (13) which penetrates into the sleeve (4) for extracting said needle (2), and angular indexing means (19) which consist of a shaping of the sleeve (4) at one of its free ends (9) according to a profile which, during extraction of the surgical needle (2) from the sleeve (4), makes it possible to direct said needle into a defined and constant position relative to the main axes of said sleeve.

2. The applicator for positioning a surgical needle which is integral with a suture thread as claimed in claim 1, characterized in that the angular indexing means (19) consist of a shaping of the external diameter (D1) of the sleeve (4) such that the inner bore (5) of said sleeve has an oblong inner profile (24) whose width (e) is less than the external diameter (D1), while its height (H) is similar to that of the external diameter (D1) so as to make it possible, during extraction of the surgical needle (2) from the sleeve (4), to direct said needle into a defined and constant position relative to the main axes of said sleeve.

3. The applicator for positioning a surgical needle which is integral with a suture thread as claimed in claim 2, characterized in that the external diameter (D1) of the sleeve (4) is deformed laterally so that the end (9) has a profile made up of two parallel flat parts (20, 21) connected to the cylindrical profile of the sleeve (4) via inclined portions (22, 23).

4. The applicator for positioning a surgical needle which is integral with a suture thread as claimed in claim 3, characterized in that the inner bore (5) has, between its oblong inner profile (24) and its internal circular diameter, an inclined portion (25) originating from the outer inclined portions (22, 23).

5. The applicator for positioning a surgical needle which is integral with a suture thread as claimed in claim 2, characterized in that the inner oblong profile (24) ends in an inner bevel (26) making it possible to position and more easily introduce the needle (2) inside the inner bore (5) of the sleeve (4) when it is necessary to retract said needle from the operating site.

6. The applicator for positioning a surgical needle which is integral with a suture thread as claimed in claim 2, characterized in that the oblong inner profile (24) communicating with the inner bore (5) of the sleeve (4) has, at the level of the inner bevel (26), a circular profile (27) of external diameter (D2) which is smaller than that (Dl) of said sleeve, giving the free end (9) what is substantially a trumpet shape.

7. The applicator for positioning a surgical needle which is integral with a suture thread as claimed in claim 2, characterized in that the profile of the internal diameter (5) of the sleeve (4) is obtained by shaping a metal tube (29).

8. The applicator for positioning a surgical needle which is integral with a suture thread as claimed in claim 2, characterized in that the profile of the internal diameter (5) is obtained by molding the sleeve (4).

9. The applicator for positioning a surgical needle which is integral with a suture thread as claimed in claim 1, characterized in that the rod (13) comprises a head (14) integral with flexible tongues (10) making it possible to limit the travel of the rod (13) relative to the sleeve (4).

10. The applicator for positioning a surgical needle which is integral with a suture thread as claimed in claim 1, characterized in that it comprises retention means (12) which consist of a plate (16) of oblong shape, of which one of the faces is integral with two clamps (17, 18) which cooperate respectively with the rod (13) and the sleeve (4).

11. The applicator for positioning a surgical needle which is integral with a suture thread as claimed in claim 10, characterized in that the sleeve (4) comprises a cylindrical head (6) provided on its outer face with a circular impression (11) which cooperates with the clamp (18) of the plate (16), while the rod (13) comprises a head (14) equipped with an impression (15) about which the clamp (17) of said plate (16) clicks into place in such a way as to maintain a constant distance (L) between the heads (6, 14) so as to ensure that the free end of the rod (13) is on the one hand in contact with the needle (2) and on the other hand at a distance (L′) from the free end (9) of the sleeve (4).

12. The applicator for positioning a surgical needle which is integral with a suture thread as claimed in claim 1, characterized in that the sleeve (4) comprises, starting from the end (9), and in the direction of the head (6), two grooves (30, 31) which are diametrically opposite one another and parallel to the longitudinal axis of the applicator.

13. The applicator for positioning a surgical needle which is integral with a suture thread as claimed in claim 12, characterized in that each groove (30, 31) has a V-shaped profile, of which the most open base, or widest base, is directed toward the end (9).

14. The applicator for positioning a surgical needle which is integral with a suture thread as claimed in claim 13, characterized in that each groove (30, 31) has a depth decreasing along the periphery of the sleeve (4) so that the deepest level is toward the free end (9) and the most open base of the V.

15. The applicator for positioning a surgical needle which is integral with a suture thread as claimed in claim 12, characterized in that the end (9) of the sleeve (4) comprises a slit (32) arranged perpendicular to the grooves (30, 31) in such a way that each groove cooperates with said slit.

16. The applicator for positioning a surgical needle which is integral with a suture thread as claimed in claim 15, characterized in that the slit (32) forms a hollow inside the sleeve (4) which communicates with the inner bore (5).
